(19) United States
(12) Patent Application Publication
(10) Pub. No.: US 2009/0247781 A1
(43) Pub. Date: Oct. 1, 2009

(54) SYNTHESIS OF PHENOXYACETIC ACID DERIVATIVES

(75) Inventors: Eric Winter, Gau-Algesheim (DE); Carsten Reichel, Rheinboellen (DE); Dieter Gutheil, Bad Kreuznach (DE)

Correspondence Address: SUGHRUE MION, PLLC, 2100 PENNSYLVANIA AVENUE, N.W., SUITE 800, WASHINGTON, DC, 20037, US

(73) Assignee: Kissei Pharmaceutical Co., Ltd., Nagano (JP)

(21) Appl. No.: 11/577,895

(22) PCT Filed: Oct. 20, 2005
(86) PCT No.: PCT/EP05/11269

§ 371 Date: Jun. 11, 2009

(30) Foreign Application Priority Data
Oct. 26, 2004 (EP) 04025432.8

(51) Int. Cl. C07C 67/03

(52) U.S. Cl. 560/42; 560/61

(57) ABSTRACT

The present invention relates to an improved process for the preparation of substituted 2-(4-carbonylmethoxy-optionally 2,5-disubstituted-phenyl-acetaldehydes, in particular 2-(4-alkoxycarbonylmethoxy-optionally 2,5-disubstituted-phenyl)-acetaldehydes and their use in the synthesis of optionally substituted 2-[4-[2-[[-2-hydroxy-2-(4-hydroxyphenyl)-1-methylethyl]-amino]ethyl]-optionally 2,5-disubstituted-phenoxy]acetic acid derivatives or the salts thereof, which may be used as pharmaceutically active substances.

The present invention relates to an improved process for the preparation of substituted 2-(4-carbonylmethoxy-2,5-disubstituted-phenyloxy)-acetaldehydes in industrial scale. In particular 2-(4-alkoxycarbonylmethoxy-disubstituted-phenyloxy)-acetaldehydes and their use in the industrial manufacture of optionally substituted 2-[4-[2-[[2-hydroxy-2-(4-hydroxyphenyl)-1-methylethyl]-amino]ethyl]-2,5-disubstituted phenoxy]acetic acid derivatives or the salts thereof is claimed. In particular the present inventions concerns the synthesis of (−)-Ethyl-2-[4-(2-{[(1S,2R)-2-hydroxy-2-(4-hydroxyphenyl)-1-methylethyl]amino}ethyl)-2,5-di-methylphenyloxy]acetate and (−)-2-[4-(2-{[(1S,2R)-2-hydroxy-2-(4-hydroxyphenyl)-1-methylethyl]amino}ethyl)-2,5-dimethylphenyloxy]acetic acid, salts thereof respectively, which may be used as pharmaceutically active substances.

The subject of the present invention is the synthesis in industrial scale of 2-[4-[2-[[2-hydroxy-2-(4-hydroxyphenyl)-1-methylethyl]-amino]ethyl]-2,5-disubstitutedphenoxy]acetic acid derivatives, which are represented by following formula I:

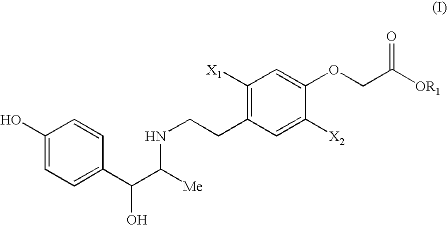

wherein R₁ is H, branched or unbranched C₁₋₆-alkyl, optionally substituted benzyl, preferably branched or unbranched C₁₋₆-alkyl, optionally substituted benzyl. C₁₋₆-alkyl preferably is methyl, ethyl, propyl, more preferably propyl, ethyl and most preferably ethyl. After manufacture of the compounds according to formula (I) R₁ can be turned into H (if it was not H before) by hydrolysis as known in the art or into NH₂, NHC₁₋₆-alkyl, N(C₁₋₁₆-alkyl)₂, with C₁₋₆-alkyl as defined above by the methods as known in the art.

X₁ or X₂ are independently from each other hydrogen, halogen or branched or unbranched C₁₋₆-alkyl.

Halogen preferably is F, Cl, Br.

X₁ or X₂ as C₁₋₆-alkyl, preferably are: methyl, ethyl or propyl, more preferably, methyl or ethyl and most preferably X₁ or X₂ each is methyl.

In the context of the present description the term “optionally substituted benzyl” shall mean that the aromatic ring system of the benzyl group may be substituted by branched or unbranched C₁₋₆-alkyl and/or C₁₋₆-alkoxyl—both of which are independently of each other optionally substituted by halo selected from the group of fluoro, chloro, bromo, jodo—in particular preferred are methyl, ethyl, trifluormethyl—1 to 6 halogens—independently selected from the group of fluoro, chloro, bromo, jodo— —CN, nitro, hydroxy, amino, optionally substituted by C₁₋₆-alkyl, in particular dimethylamino or diethylamino.

The preferred compounds to manufacture according to the present invention are

### 1) X₁═Br, X₂═H, R₁═H

### 2) X₁═Cl, X₂═H, R₁═H

### 3) X₁═C₁, X₂═Cl, R₁═H

### 4) X₁═H, X₂═H, R₁═H

### 5) X₁═C₁, X₂═H, R₁═H

### 7) X₁═Cl; X₁═Cl, R₁=Et

### 8) X₁ Me; X₁=Me, R₁=Et

### 9) X₁=Me; X₁=Me, R₁═H.

Preferred stereospecific details are given in formula (II):

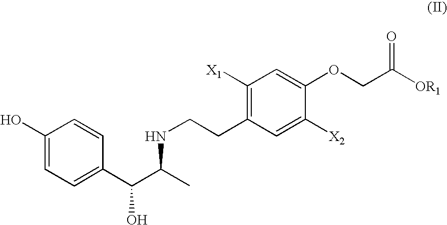

wherein X₁, X₂ and R₁ are defined as above, with all preferences as above (in particular compounds 1 to 9).

The claimed compounds according to formula (II) can be named as R₁—(−)-2-[4-[2-[[(1S,2R)-2-hydroxy-2-(4-hydroxyphenyl)-1-methylethyl]-amino]ethyl]-2-X₂,5-X₁ phenoxy]acetates, the corresponding acetic acid derivative (R₁=H) or a pharmacologically acceptable salts of any of them.

The compounds of formula (I) are known from EP 1 095 932, SP-2002-338513 and other publications. They have a β₃-adrenergic receptor-stimulating effect (β₃-adrenergic receptor agonists) and are interesting as agents for preventing or treating obesity, adiposis, hyperglycemia, diseases caused by intestinal hypermotility, diseases caused by intestinal hyperkinesia, pollakiuria, urinary incontinence, depression, diseases caused by biliary calculi or hypermotility of the biliary tract and cholelithiasis. Among the most preferred indication areas are urinary incontinence be it in form of overactive bladder, stress urinary incontinence, urge urinary incontinence or mixed forms thereof.

For the sake of clarity and completeness a certain terminology will be used hereinafter. The compounds according to general formula (I) shall include the embodiment described expressis verbis as well as all chemical or pharmacological equivalents. The compounds can be turned into pharmacologically acceptable salts thereof. Examples of pharmaceutically active salts for each of the compounds which are the subject of this description include, without being restricted thereto, salts which are prepared from pharmaceutically acceptable acids, including organic and inorganic acids. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, famaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like.

It will be appreciated that in the course of the synthetic route there is made use of a new class of compounds as key intermediate, which also is subject of the present invention. Such compounds are 2-(4-(substituted carbonyl)methoxy-2,5-disubstituted-phenyl)-acetaldehydes, which are another objective of the present invention. They are represented by structure V.

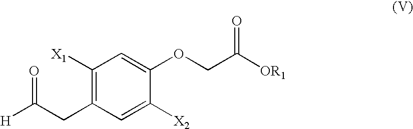

wherein R₁ and X₁ and X₂ are as defined above.

Methods for preparing compounds according to formula (I) are disclosed in EP 1 095 932 and JP-2002-338513. The synthetic route disclosed in JP-2002-338513 requires 5 steps starting from a compound of formula

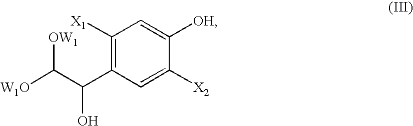

wherein X₁ and X₂ both are methyl. W₁ is an alkyl group. Up to the recrystallised end product of formula (I) three isolated intermediates are passed, which are acetals or semiacetals and which have been found to be sensitive to certain physical properties like temperature and which make a technical manufacturing process in industrial standard complex and difficult.

Accordingly, it is one objective of the present invention to improve the synthesis known from the prior art.

It is another aspect of the present invention to present a manufacturing process for to produce a compound of formula (I) in high amounts in industrial standard.

It is another objective of the present invention to present a manufacturing process for to produce a compound of formula (I) which passes stable with long shelf-life time.

Another objective is to create a manufacturing process with good manufacturing properties. It is another objective of the present invention to create a manufacturing process with a reduced number of steps and finally with optimised yields of the products.

The objectives are met by the method according to the invention, because the new route comprises only 3 steps from the compound of formula (III) up to the end product (formula I) and creates stable intermediates, for which storage is uncomplicated.

Additionally, the inventive synthetic routs allow the production of a compound according to formula (I) in high amounts and in industrial standard.

Scheme 1 gives an overview about the method of preparation according to the invention:

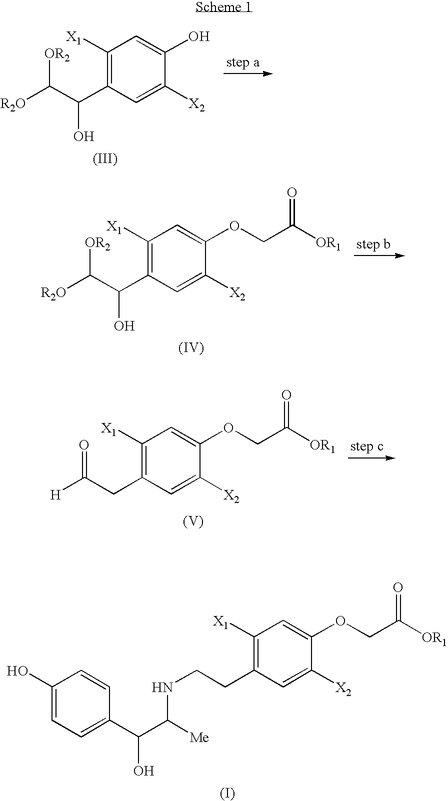

In the above scheme is

R₁ preferably is branched or unbranched C₁₋₆-alkyl or H; preferably it is C₁₋₆-alkyl, among which methyl, ethyl and propyl are preferred. More preferred are propyl and ethyl and most preferred is ethyl;
R₂: independently of each other is branched or unbranched C₁₋₆-alkyl or both R₂ together are a 5- or 6 membered saturated ringsystems such as 1,3-Dioxanyl or 1,3 Dioxolanyl; preferably it is C₁₋₆-alkyl, among which methyl, ethyl and propyl are preferred. More preferred are methyl and ethyl and most preferred is methyl;
X₁ or X₂ independently from each other are as defined above, preferably C₁₋₆-alkyl, among which methyl, ethyl and propyl are preferred. More preferred are methyl and ethyl and most preferred is methyl.

Another aspect of the invention is the synthesis of compounds of formula (II) starting from compounds of formula (IV) (step b) as well as intermediate V itself.

Compounds of formula (III), are available over the method described in JP-2002-338513 for example. In particular, example 1 of JP-2002-338513 describes the synthesis of the compound of formula (III), wherein X₁ and X₂ as well as R₂ are methyl, for which the present synthetic rout can be applied as well.

The different steps of the method according to the invention as outlined in scheme 1 may be carried out according to procedures known per se, particularly according to the following procedures. For the sake of clarity it shall be pointed out that the building blocks and intermediates used as herein described may be varied according to the knowledge of the state of the art to finally achieve the same final product. Such modifications include but are not limited to masking one or more groups which are not intended in the particular step by the reversible introduction of an appropriate protecting group or a reversible transformation of said group and the like. The present invention refers to such alternatives and equivalents which for the skilled person in the art are easy to be achieved.

### Step a:

The phenoxyacetic acid ester derivatives represented by the above general formula (IV) can be prepared by reacting a phenol derivative of general formula (III) with a compound of formula (IV)

ZCH₂CO₂R₁ (VI),

wherein Z represents a substitution group such as a halogen atom, for example a chlorine or bromine, tosylate, CO2R₁, wherein R₁ is as defined above.

The preferred reaction conditions comprise an inert solvent, and/or a temperature of 0 to 100° C. and/or a reaction time of 1 to 24 hours. In case of Z being an halogen, catalytic amounts of sodium iodide may be added to the reaction mixture.

The inert solvents, which are suitable for this reaction, include for example ethers such as tetrahydrofuran, ketones such as acetone and methyl ethyl ketone, acetonitrile, N,N-dimethylformamide, N,N-dimethylacetamide and their mixtures. The mixtures may contain two or more of the above-mentioned solvents. As base inorganic or organic bases may be used. As example of inorganic bases are named: sodium or potassium hydroxide, sodium carbonate, potassium carbonate and cesium carbonate, as examples of organic bases are named triethylamine or ethyl-diisopropylamine. The reaction also may be carried out under phase transfer conditions. Usually, 1 to 5 equivalents of the compound of general formula (VI) and of the base per equivalent of the compound of general formula (III) are used. As for the molar ratio of the compound of formula (VI) and of the base, although they are usually used in equimolar amounts, either of them may be used in excess.

After the completion of the reaction, the reaction product is extracted and concentrated by ordinary methods to obtain the desired phenoxyacetic acid ester derivative of the general formula (A). The phenoxyacetic acid ester derivative (IV) may be purified before entering the subsequent step, but it is also possible to use it in the next step without purification.

In a preferred variation of step a, the compound of formula (III) is reacted with about 1.2 equivalents of a compound of the above general formula VI, wherein Z is a bromine atom, in the presence of about 1.3 equivalents of potassium carbonate and catalytic amounts of sodium iodide in acetone for about 3 hours under reflux to yield the compound of formula IV.

### Step b:

The phenoxyacetic acid ester derivatives of above general formula (V) are then transformed into the aldehydes of general formula (V) by transforming the acetal into the aldehyde while simultaneous and/or subsequent reduction of the hydroxyl group.

The reduction of the hydroxyl group may be performed by transforming the hydroxyl group of the compound of formula (V) into a leaving group, for example by reacting the compound of general formula (IV) with a trialkylhalosilane such as trimethylchlorosilane, methyldiphenylchlorosilane, tert-butyl-dimethylchlorosilane or tert-butyl-diphenylchlorosilane or the like to give the corresponding trialkylsilyloxy derivative. Such silyl-group can be cleaved subsequently under reductive conditions. For the silylation, 2 to 5 equivalents of the trialkylhalosilane can be used, the use of about 3.1 equivalents being preferred. Additionally, sodium iodide may be added in an amount similar to that of the trialkylhalosilane. Suitable solvents for the reaction include but are not limited to acetonitrile, which is preferred. The reaction is usually carried out at a temperature between −50 and +25° C., preferably between −40 and 0° C., most preferably between −15 and −25° C., in particular at about −20° C. The reaction time may vary between 1 and 24 hours, often, 1-3 hours, in particular about 2 hours will be enough for completion of the reaction. The reaction mixture then may be washed with aqueous solutions of sodium acetate and sodium thiosulfate. After the completion of the reaction, the reaction product is extracted and concentrated by ordinary methods.

Before the removal of the dimethoxy group the residue so obtained may optionally be charcoaled using a suitable solvent such as tetrahydrofuran, dioxane, methanol, ethanol, toluene or the like. The purified solution thus obtained or the unpurified residue dissolved in one of the solvents listed as suitable for charcoiling is then treated with water and oxalic acid, perchloro acid, sulphuric acid, hydrochloric acid, p-toluene sulfonic acid for several hours at room temperature. In general, 1-10 equivalents of oxalic acid are use; about 3.4 equivalents being preferred. The work up is done by ordinary methods.

### Step c:

For to make the final product, the aldehyde of general formula (II) is reacted with the corresponding amine, preferably 4-hydroxy-norephedrine (HNE), an amine having the following structure

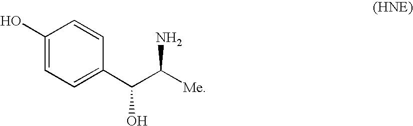

In alternative routes an enantiomer or diasteromer of the compound can be used as well as a racemic form, whereby it is noted that two chiral centres are present in SINE. In case of a racemic form of HNE, racemic separation may be performed in a subsequent step to complete the manufacture of the preferred final product of (1S,2R) configuration. It is also possible to protect the OH-group(s) by an appropriate protecting group such as disclosed in the state of the art.

The coupling reaction of (V) and the amine (HNE preferably) is done in the presence of a reducing agent in an inert solvent. The temperature is preferably kept between −20 and 60° C. until completion or stop of the reaction. The reaction time usually is between 1 and 48 hours.

Suitable reducing agents include alkali metal borohydrides such as NaBH₄, NaCNBH₃, NaBH(OAc)₃ and NaBH(OMe)₃, and borane compounds such as BH₃ •pyridine and BH₃•N,N-diethylamine. If necessary, they can be used in the presence of an acid such as acetic acid, p-toluenesulfonic acid, methanesulfonic acid, sulphuric acid or hydrochloric acid or a base such as triethylamine. Furthermore, a catalytic amount of a metallic catalyst such as 5 to 10% palladium carbon, Raney nickel, platinum oxide, palladium black or 10% platinum carbon (sulphur-poisoned) can be used in a hydrogen atmosphere. When an alkali metal borohydride of a borane is used as the reducing agent, the amount thereof is suitably selected in the range of 0.5 to 5 equivalents per equivalent of the aldehyde of formula V. The inert solvents which can be used for this reaction include, for example, ethers such as tetrahydrofuran, 1,2-dimethoxyethane and dioxane, halogenated hydrocarbons such as methylene chloride and 1,2-dichloroethane, organic carboxylic acids such as acetic acid, hydrocarbons such as toluene, alcohols such as methanol and ethanol, and acetonitrile. These solvents can be used either alone or in the form of a mixture of two or more of them. After the completion of the reaction, the insoluble matter is removed, if necessary, and the product is extracted and concentrated by ordinary methods to obtain the desired phenoxyacetic acid derivative of formula I.

The preferred reducing agent is Pd/C under a hydrogen atmosphere, particularly at a concentration of 10%. Tetrahydrofuran is preferred as solvent.

The phenoxyacetic acid derivative of formula (I) can be converted into a physiologically acceptable salt thereof, in desired, by an ordinary method. The salts include acid addition salts thereof with inorganic acids such as hydrochloric acid, hydrobromic acid, hydroiodic acid, sulphuric acid and phosphoric acid as well as acid addition salts thereof with organic acids such as formic acid, acetic acid, methanesulfonic acid, benzenesulfonic acid, p-toluenesulfonic acid, propionic acid, citric acid, succinic acid, tartarc acid, fumaric acid, butyric acid, oxalic acid, malonic acid, maleic acid, lactic acid, malic acid, carbonic acid, glutamic acid and aspartic acid. By preference, the hydrochloric acid addition salt of the compound of formula (I) is prepared.

### Optional Step d:

Optionally, for further purification, the compound of general formula (I) or its acid addition salt thus obtained may be recrystallised using suitable solvents. Such suitable solvents include alcohols such as methanol, ethanol, butanol, t-butanol or isopropanol and ethers such as methyl tert-butyl ether or diethyl ether.

In a preferred variation of step d, the hydrochloride of the compound of formula (I) is recrystallised from a mixture containing 40 vol-% of ethanol and 60 vol-% of methyl tert-butyl ether. The isolated crystals are washed with ice-cold mixtures of ethanol and methyl tert-butyl ether with a even larger amount of methyl tert-butyl ether than in the mother liquor and subsequently with methyl tert-butyl ether alone.

Yet another optional step 1st step e which is transforming the product according to step c or d into a salt form, if it is not already the whished salt. To do so it is referred to the prior art, in particular to the one as disclosed above.

As mentioned above the compounds according to formula (I) or (II) with R₁ being alkyl can optionally be turned into the free acid by hydrolysation methods or into an amide by amination methods as known in the art.

The advantage of the present invention over the prior art in particular are:
significantly improved overall yield in a chemical process of industrial scale,
avoidance of instable intermediates such as semiacetals,
the process can be shortened by one step
better capacity—time yield. The term capacity refers to the capacity of the reaction vessel in cubic meter, the term time to the reaction time needed to manufacture 1 kg of substance.

The major improvement is a better overall yield which is of high importance in particular for a chemical process of industrial scale.

The following examples are intended to illustrate the invention in greater detail:

### EXAMPLE 1

### Ethyl 2-[4-(2,2-dimethoxy-1-hydroxyethyl)-2,5-dimethylphenoxy]acetate

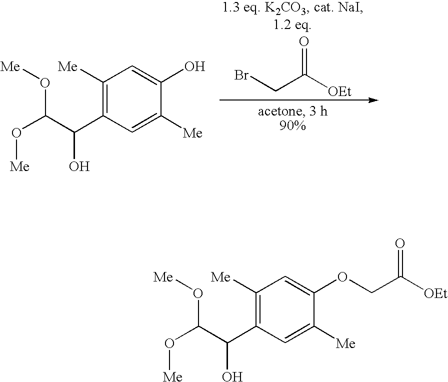

4-(2,2-Dimethoxy-1-hydroxyethyl)-2,5-dimethylphenol (20.0 g, 88.3 mmol, 1.0 eq.), K₂CO₃ (15.9 g, 115 mmol, 1.3 eq.), ethyl bromoacetate (17.7 g, 106 mmol, 1.2 eq.) and NaI (cat. amount) are mixed in acetone (20 ml) at room temperature. The suspension is stirred and refluxed for 3 h. After adding triethylamine (5 ml, 35 mmol, 0.4 eq.) the mixture is diluted with toluene (150 ml) and washed with aq. NaOH (0.5 M, 100 ml) and water (100 ml). The organic phase is concentrated to an oily residue and cyclohexane (400 ml) is added at 55° C. After cooling down to 0° C. the white crystals are filtered off washed with cyclohexane (60 ml) and dried at 45° C. i. v.

Yield: 24.8 g, 79.5 mmol, 90%

Melting point: 83° C.

### EXAMPLE 2

### 2-(4-Ethoxycarbonylmethoxy-2,5-dimethyl-phenyl)-acetaldehyde

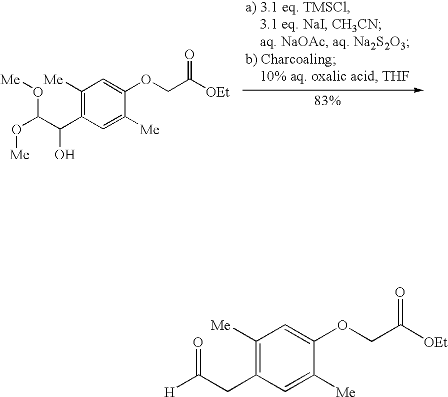

NaI (29.8 g, 197 mmol, 3.1 eq.) and trimethylchlorosilane (21.6 g, 197 mmol, 3.1 eq.) are stirred at 5° C. in acetonitrile (50 ml) for 15 min., then the suspension is cooled down to −20° C. A solution of ethyl 2-[4-(2,2-dimethoxy-1-hydroxyethyl)-2,5-dimethylphenoxy]acetate (20.0 g, 63.0 mmol, 1.0 eq.) in acetonitrile (50 ml) is added and the mixture is stirred for 2 h. For workup, aq. NaHCO₃ (150 ml) and sat. aq. Na₂S₂O₃ (90 ml) are added and the mixture is diluted with toluene (140 ml) and warmed up to 5° C. The organic phase is separated and washed with aq. Na₂S₂O₃ (40 ml) and water (2×40 ml). The solvent of the organic phase is distilled off i. v. completely and the oily residue is diluted with THF (50 ml). The solution is charcoaled and after filtration the organic phase is treated with water (170 ml) and oxalic acid (20.0 g, 218 mmol, 3.4 eq.). The reaction is complete after 3.5 h and toluene (140 ml) is added. After phase separation the organic phase is washed with water (2×40 ml), aq. NaHCO₃ (40 ml) and again water (2×40 ml). Finally, the crude product is concentrated.

Yield: 13.1 g, 52.3 mmol, 83%

### EXAMPLE 3

### Ethyl (−)-2-[4-[2-[[(1S,2R)-2-hydroxy-2-(4-hydroxyphenyl)-1-methylethyl]-amino]ethyl]-2,5-dimethylphenoxy]acetate hydrochloride

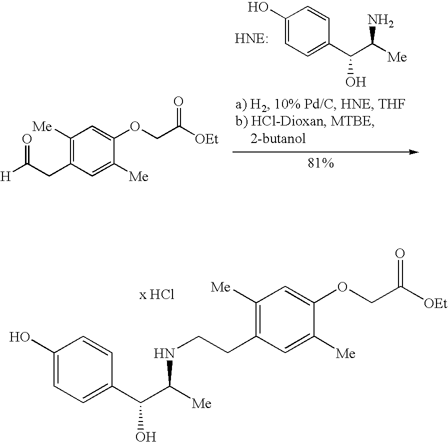

2-(4-Ethoxycarbonylmethoxy-2,5-dimethyl-phenyl)-acetaldehyde (30.0 g, 120 mmol, 1.1 eq.), 4-hydroxy-norephedrine (18.4 g, 110 mmol 1.0 eq.) and Pd/C (10% Pd, 50% water, 7.6 g) are mixed in tetrahydrofuran (THF) (300 ml) at room temperature, then 12 is bubbled through the suspension until the reaction is finished. After filtration, concentration and washing with toluene (300 ml) the organic phase is washed with water (3×150 ml). The solution is concentrated and 2-butanol (60 ml) is added. At 70° C. HCl (˜1.5 mol/l in 1,4-dioxane, 60 ml, 0.85 eq.) is dropped to the reaction mixture, and the suspension is cooled down to 50° C. Then, methyl tert-butyl ether (300 ml) is added slowly. The crystals are stirred overnight, filtered off, washed with ethanol/methyl tert-butyl ether (1:5, 60 ml) and methyl tert-butyl ether (60 ml) and dried at 75° C. i. v.

Yield: 39.0 g, 89.0 mmol, 81%.

Melting point: 176° C.

### EXAMPLE 4

### Recrystallisation of Ethyl (−)-2-[4-[2-[[(1S,2R)-2-hydroxy-2-(4-hydroxyphenyl)-1-methyl-ethyl]-amino]ethyl]-2,5-dimethylphenoxy]acetate hydrochloride

Ethyl (−)-2-[4-[2-[[(1S,2R)-2-hydroxy-2-(4-hydroxyphenyl)-1-methylethyl]-amino]ethyl]-2,5-dimethylphenoxy]acetate hydrochloride (e.g. from example 3) (20.0 g, 45.6 mmol) is solved in ethanol (110 ml) at 7° C. The clear solution is cooled to 58° C. and methyl tert-butyl ether (72 ml) is added slowly. After cooling down to 0° C. the crystals are filtered off, washed with ice-cold ethanol/methyl tert-butyl ether (1:5, 50 ml) and methyl tert-butyl ether (50 ml). The white crystals are dried at 70° C. i. v.

Yield: 16.6 g, 37.9 mmol, 83%

Melting point: 196-197° C.

Other compounds can be made accordingly. Preferred is the synthesis of the compounds according the examples.

1. Method for the preparation of compounds of general formula 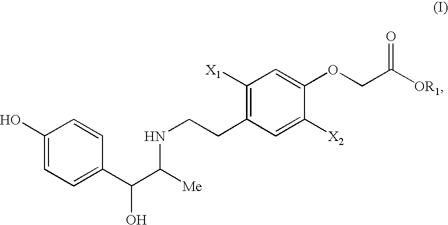
or a salt thereof, wherein R₁ is branched or unbranched C₁₋₆-alkyl or H; preferably being branched or unbranched C₁₋₆-alkyl, among which methyl, ethyl, propyl are preferred, more preferred are propyl, ethyl and most preferred is ethyl;
X₁ or X₂ are independently from each other C₁₋₆-alkyl, preferably methyl, ethyl, propyl, more preferably methyl, ethyl and most preferably methyl,
comprising the following steps:
a) reacting a compound of general formula 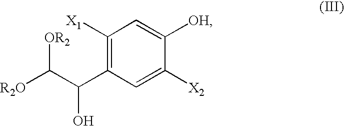
wherein X₁ or X₂ are as defined above and R₂ are independently of each other is branched or unbranched C₁₋₆-alkyl preferably being methyl, ethyl, propyl, more preferably methyl, ethyl and most preferably methyl; or both R₂ together are a 5- or 6 membered saturated ring system such as 1,3-Dioxanyl or 1,3 Dioxolanyl, with a compound of general formula (VI)
ZCH₂CO₂R₁ (VI),
wherein Z represents a substitution group and R₁ is as defined above,
b) transforming the compound according the aforementioned step, which is represented by general formula (IV) 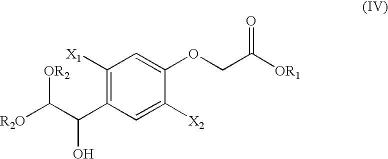
with R₁, R₂, X₁ and X₂ as defined above into an aldehyde of general formula (V) 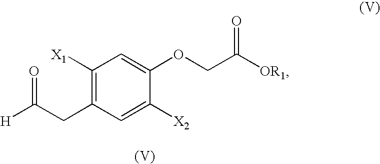
wherein R₁, R₂, X₁ and X₂ defined above by
i) transforming the free hydroxyl group of a compound of general formula (IV) into a leaving group and
ii) removing the previously generated leaving group under reductive conditions,
c) reacting the aldehyde of formula (V) thus obtained with 1-(4-hydroxy-phenyl)-1-hydroxy-2-propylamin, preferably, 4-hydroxy-norephedrine to give the compound of general formula I, and
d) optionally recrystallising the product thus obtained and/or
c) optionally transforming the product into a pharmacologically acceptable salt form as the hydrochloride.

2. Method according to claim 1, wherein R₁, R₂, X₁ and X₂ each represent a linear or branched C₁₋₃-alkyl group.

3. Method according to claim 1, wherein R₁ is ethyl and R₁, R₂, X₁ and X₂ each are methyl.

4. Method according to claim 1, wherein Z is a chlorine or bromine atom and step a) is carried out in the presence of a base.

5. Method according to claim 4 wherein the base is selected from the group consisting of sodium carbonate, potassium carbonate and cesium carbonate.

6. Method according to claim 4, wherein step a is performed in the presense of catalytic amounts of sodium iodide.

7. Method according to claim 1, wherein the leaving group of step b) is a trialkylsilyloxy group.

8. Method according to claim 1, wherein step c) is carried out in the presence of a reducing agent.

9. Method according to claim 8, wherein the reducing agent is selected from the group consisting of alkali metal borohydrides, borane compounds and hydrogen atmosphere in the presence of a metallic catalyst.

10. Method according to claim 9, wherein Pd/C under a hydrogen atmosphere is used as reducing agent.

11. Method according to claim 1, wherein step c involves addition or presence of an acid, preferably hydrochloric acid, and step e is not applied.

12. Method according to claim 1, wherein step d the compound of formula I, preferably (−)-Ethyl-2-[4-(2-{[1S,2R)-2-hydroxyphenyl)-1-methylethyl]amino}ethyl)-2,5-dimethylphenyloxy]acetate Hydrochloride, is optionally recrystallised in a mixture of ethanol and methyl butyl ether.

13. Method according to claim 1, wherein the compound of formula 1, preferably (−)-Ethyl-2-[4-(2-{[1S,2R)-2-hydroxyphenyl)-1-methylethyl]amino}ethyl)-2,5-dimethylphenyloxy]acetate, is optionally recrystallised and the isolated product is transformed into a salt, preferably into the hydrochloride.

14. Method according to claim 1 comprising any of steps b, c and optionally d and/or e.

15. Method according to claim 1 for the preparation of a compound of formula 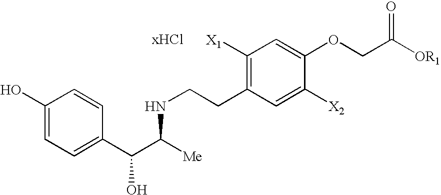
wherein R₁ is ethyl, R₂, X₁ and X₂ each are methyl and for step a—if applied—Z represents a chlorine or bromine atom, whereby reaction step a is carried out in the presence of a base such as potassium carbonate and optionally catalytic amounts of sodium iodide, in step b the hydroxyl group of a compound of general formula (v) is converted into a trimethylsilyloxy group as leaving group,
and in step c) the aldehyde according to formula (II) is dissolved in THF and coupled with HNE under a hydrogen atmosphere and in the presence of PC/C with 4-hydroxy-norephedrine, using a solution comprising HCl in 1,4-dioxane for the work-up, to give the desired product as shown above, and
d) optionally recrystallising the product thus obtained using a mixture of ethanol and methyl tert-butyl ether as solvent.

16. Method for the preparation of compounds of general formula (V) 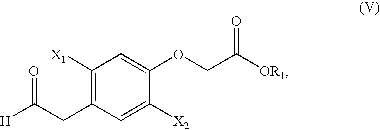
wherein R₁ is branched or unbranched C₁₋₆-alkyl or optionally substituted benzyl, preferably C₁₋₆-alkyl preferably methyl, ethyl, propyl, more preferably methyl, ethyl and most preferably ethyl and X₁ or X₂ are independently from each other hydrogen, halogen or branched or unbranched C₁₋₆-alkyl, preferably methyl, ethyl, propyl, more preferably methyl, ethyl and most preferably methyl, in particular the compound according to formula (Va) 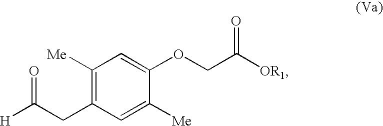
comprising the following steps:
a) transforming the free hydroxyl group of a compound of general formula 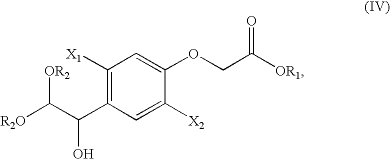
wherein R₁, X₁ and X₂ are as defined above and R₂ are independently from each other a linear or branched C₁₋₆-alkyl group, into a leaving group and
b) removing the previously generated leaving group under reductive conditions.

17. Method according to claim 16, wherein R₁, X₁, X₂ and R₂ independently from each other represent a linear or branched C₁₋₃-alkyl group.

18. Method according to claim 17, wherein R₁ is ethyl and X₁, X₂ and R₂ are each methyl.

19. Method according to claim 16, wherein the leaving group is a trialkylsilyloxy group.

20. A compound of general (V) 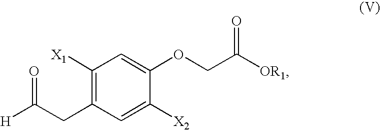
wherein R₁ is a linear or branched C₁₋₆-alkyl or H, preferably C₁₋₆-alkyl, among which C₁₋₃-alkyl is preferred, among which methyl, ethyl, propyl are more preferred and among which methyl, ethyl are in particular preferred and ethyl is the most preferred and X₁ or X₂ are independently from each other C₁₋₆-alkyl, preferably methyl, ethyl, propyl, more preferably methyl, ethyl and most preferably methyl.

21. Compound according to claim 20, wherein R₁ is a linear or branched C₁₋₃-alkyl group and X₁ and X₂ each are methyl.

22. Compound according to claim 20, represented by formula (Va) 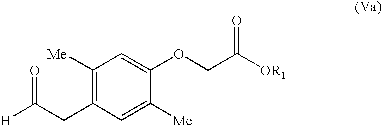
wherein R₁ is ethyl.

23. Methods for the preparation of compounds of general formula (I) 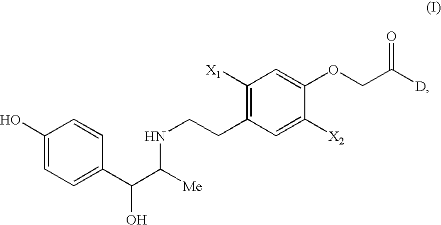
with X₁ and X₂ as defined in claim 1 and D being OH or NH₂, NHC₁₋₆-alkyl, N(C₁₋₆-alkyl)₂, with C₁₋₆-alkyl as defined in claim 1 by applying a method as defined in claim 1 and subsequent hydrolysis or amination by NH₂, NHC₁₋₁₆-alkyl, N(C₁₋₆-alkyl)₂.
